(19) United States
(12) Patent Application Publication
(10) Pub. No.: US 2006/0088580 A1
(43) Pub. Date: Apr. 27, 2006

(54) Hormone-containing transdermal therapeutic system with an active substance reservoir based on vinylacetate-vinylpyrrolidone copolymer with improved cohesion

(76) Inventors: Reinhold Meconi, Neuwied (DE); Robert-Peter Klein, Neuwied (DE); Ursula Hildegard Klein, Neuwied (DE); Frank Seibertz, Rheinbrohl (DE); Franz-Josef Becker, Neuwied (DE); Ursula Cotte, Unkel (DE)

Correspondence Address: BIRCH STEWART KOLASCH & BIRCH, PO BOX 747, FALLS CHURCH, VA, 22040-0747, US

(21) Appl. No.: 10/507,628

(22) PCT Filed: Mar. 12, 2003
(86) PCT No.: PCT/EP03/02521

§ 371 Date: Oct. 7, 2005

(30) Foreign Application Priority Data
Mar. 16, 2002 (DE) 102 11 832.9

(51) Int. Cl. A61K 9/70

(52) U.S. Cl. 424/449

(57) ABSTRACT

A transdermal therapeutic system in patch form for the controlled delivery of active substances to the human or animal skin comprises a backing layer, an active substance-containing reservoir attached thereto and a detachable protective layer, is characterized in that the active substance-containing reservoir contains as main components thereof at least one film former as well as at least one polymer which prevents or suppresses the crystallisation of the active substance or active substances.

The present invention relates to a transdermal therapeutic system (TTS) in the form of a patch for the controlled release of active substances to human or to animal skin, wherein the recrystallisation of the active substances is prevented or inhibited.

The transdermal administration of pharmaceutical active substances is useful especially in those cases where after oral administration a large portion of the active substance is metabolized during the first passage through the mucous membranes of the gastrointestinal tract, or is retained by the liver (first pass effect), and/or where the active substance has a low plasma half time. On the other hand, it is a prerequisite of transdermal administration that the administration form utilized enables a delivery of the active substance which is as high as possible and lasts for an extended period. Here, the highest possible active substance delivery rates (flux rates) through the skin should be achieved in order to build up and maintain a sufficiently high plasma level for the desired therapeutic effect to occur.

If the transdermal delivery rate obtained is too low, the surface of the active substance-containing patch via which the active substance delivery to the skin takes place must be enlarged correspondingly to enable the administration of therapeutically active doses nevertheless. On the other hand, the enlargement of the delivery surface constitutes a drawback since with large-area systems there is a risk of not achieving a complete skin contact so that the active substance delivery is disturbed. In addition, small-area patches are preferred by the patients.

The active substance delivery rate is dependent on the one hand on the permeability characteristics of the skin for the active substances concerned, and on the other hand on the concentration of the active substances in the matrix of the transdermal therapeutic system.

The permeability characteristics of the skin can be improved by permeation enhancers (enhancers); substances suitable for this purpose are in principle known to those skilled in the art.

To maximize the active substance release it is common practice to increase the concentration of active substance in the active substance reservoir until the saturation concentration is reached or even exceeded, in order to thereby increase the thermodynamic activity of the active substance.

As a consequence, however, there is a high probability that during storage or during the time of application a recrystallization of the active substance occurs in the active substance matrix as a result of the saturation concentration being exceeded. This phenomenon is known to occur, in particular, in estradiol-containing patches. As a result of the recrystallization, the thermodynamic activity of the active substance decreases strongly, and as a consequence also the active substance delivery rate. For this reason, several solutions have been proposed in the prior art by which it is possible to achieve high active substance concentrations in the active substance reservoir of the patch and which are at the same time to prevent the recrystallization of the active substance.

For example, from U.S. Pat. No. 4,624,665 there are known systems containing the active substance in the reservoir in microencapsulated form. The structure and manufacture of these systems is very complicated as the active substance is microencapsulated and has to be homogeneously distributed in a liquid phase which must then be embedded in a further operation between the backing layer and the membrane.

EP 0 186 019 A1 describes active substance patches wherein water-swellable polymers are added to a caoutchouc/adhesive resin composition, and from which estradiol can be released. It has turned out, however, that the release of estradiol from these active substance patches is by far too low and does not meet the therapeutic requirements. In DE-OS 39 33 460 there are described active substance patches on the basis of homopolymers and copolymers with at least one derivative of acrylic or methacrylic acid, which patches are in addition to contain water-swellable substances.

DE-OS 195 00 662 describes a transdermal therapeutic system comprising an ethyl cellulose-based estradiol-containing active substance reservoir with a high content of colophony esters as tackifying resin, along with up to 20%-wt. of lauric acid, which lauric acid is to counteract the recrystallisation of the active substance and thereby to counteract the decrease in its delivery rate.

In the literature, several other substances are described which act as crystallization inhibitors and are to prevent the crystallization of estradiol in particular, these are, for instance, silicon dioxide (U.S. Pat. No. 5,676,968) or water-free glycerol (WO 96/05814). The addition of such substances frequently entails drawbacks, it can, for instance, impair the mechanical properties (cohesion) of the patch or lead to problems in the manufacture of the patches.

It was therefore the object of the present invention to provide a transdermal therapeutic system which has a simple structure and can be manufactured in a cost-effective manner, and which is capable of delivering pharmaceutical active substances at high delivery rates to the skin, whereby skin permeation rates are to be achieved which are far above the permeation rates obtainable by known systems but are in any case sufficient for therapeutic purposes or for contraception, without the surface dimensions of the patch becoming unacceptably large.

Surprisingly, it has turned out that transdermal therapeutic systems (TTSs) in patch form which have the structure described in the introductory part of claim 1 enable very high skin permeation rates for active substances if the active substance-containing reservoir contains as main components at least one film former as well as at least one polymer which prevents or at least suppresses the crystallization of the active substances(s).

Further advantageous embodiments of the transdermal therapeutic system according to the invention are described in the subclaims.

With a transdermal therapeutic system according to the invention which contained the active substances estradiol and norethindrone acetate, skin permeation rates were achieved that were several times higher than those achieved by the reference product Evorel Conti. Both for the estradiol permeation and for the norethindrone acetate permeation, values were obtained that were four times the value of the respective value achieved by the reference product Evorel Conti.

Because of this increase in skin permeation, it is now possible to provide active substance-containing skin patches which have a very small surface area.

As film former, which according to claim 1 is contained in the reservoir as a main component, there is preferably used a substance selected from the group comprising derivatives of cellulose, polymethyl methacrylates, and polyacrylates. Among the cellulose derivatives ethyl cellulose, hydroxypropyl cellulose and hydroxypropylmethyl cellulose are especially preferred. Combinations of different film formers can be used too. The portion of the film former(s) preferably amounts to 10 to 50%-wt, relative to the active substance-containing reservoir.

The TTSs according to the invention contain as a further main component of the active substance-containing reservoir at least one polymer preventing the crystallization or of the active substance(s); the content of this polymer or the polymers amounts to 10 to 50%-wt, relative to the active substance-containing reservoir. As a crystallization-inhibiting polymer an ethylene-vinyl acetate-vinylpyrrolidone copolymer is used with preference.

The systems according to the invention are characterized by a certain water absorptivity; preferably, the active substance-containing reservoir is able to absorb, respectively contain, at least 15%-wt of water, especially preferred at least 20%-wt.

The active substance concentration, relative to the active substance-containing reservoir, is dependent on the active substance utilized in each particular case, and is preferably in the range of 0.5 to 20%-wt, relative to the active substance-containing reservoir.

Furthermore, the active substance-containing reservoir may have a content of at least one enhancer; “enhancer” here meaning a substance which improves the skin permeation of the active substances to be administered. The enhancer or enhancers is/are added in a concentration of 0.5 to 50%-wt, relative to the active substance-containing reservoir. The enhancer or enhancers is/are preferably selected from the group containing the following substances: lauric acid diethanolamide (e.g. Comperlan LD), oleic acid diethanolamide (e.g. Comperlan OD), coconut fatty acid diethanolamide (e.g. Comperlan COD), D-alpha-tocoperol (e.g. Copherol), lauric acid hexyl ester (e.g. Cetiol A), 2-octyl dodecanol (e.g. Eutonal) and dexpanthenol.

According to a further embodiment of the invention it is provided that emulsifiers or plastifiers are added to the active substance-containing reservoir in a concentration of up to 10%-wt, preferably of 0.1 to 5%-wt. Substances which are suitable as plasticizers or emulsifiers are in principle known to those skilled in the art.

It may also be advantageous to add tackifying resins to the active substance-containing reservoir to improve the adhesive properties of the reservoir on the skin. If necessary, fillers can also be added to the active substance reservoir.

It may prove particularly advantageous for the active substance-containing reservoir to be composed of two or more layers. In this case, the individual layers may contain different active substances or active substance concentrations, or have a different polymer composition, or they may differ in their composition in another way. Furthermore, there may be inserted a flat-shaped body between the individual layers of the active substance-containing reservoir which may be, for instance, a membrane, a film, a textile woven fabric, a textile material or a nonwoven. A further embodiment consists in that the TTS according to the invention is provided with an additional pressure-sensitive adhesive layer and/or a pressure-sensitive adhesive margin or edge; this is useful especially if the tackiness of the active substance-containing matrix appears to be insufficient.

The TTSs according to the invention are characterized by a small layer thickness; preferably, the layer thickness of the active substance-containing reservoir is 0.02 mm to 0.5 mm, especially preferred 0.03 to 0.2 mm.

The structure of the TTSs according to the invention comprises—apart from an active substance reservoir—an active substance-impermeable backing layer, as well as a likewise active substance-impermeable, detachable backing layer. Suitable as a backing layer are, above all, poly-esters which are characterized by a particularly high strength, but also almost any other well-tolerated plastics such as polyvinyl chloride, ethylene vinyl acetate, vinyl acetate, polyethylene, polypropylene, cellulose derivatives, or combinations of different films, and many more. In a given case, the backing layer may be provided with an additional coat, e.g. by vapour deposition with metals or other diffusion-blocking additives such as silicon dioxide, aluminium oxide, or similar substances known to those skilled in the art. For the detachable protective layer, the same materials can be used as for the backing layer, provided that the protective layer is rendered detachable by appropriate surface treatment such as, for example, siliconisation. But other detachable protective layers, such as polytetrafluoroethylene-treated paper, cellophane, polyvinyl chloride or the like may be used as well.

Because of the reduced tendency for recrystallisation of the active substance, the TTSs according to the invention enable comparatively high active substance delivery rates, and are therefore excellently suited for transdermal administration of active substances, especially for the prophylaxis and therapy of diseases in humans or in veterinary medicine.

The following examples describe TTSs which are of a composition in accordance with claim 1, and the skin permeation rates achieved thereby.

### EXAMPLE 1

30.8 g of ethylene-vinylacetate-vinylpyrrolidone copolymer (Plasdone S 630, from International Speciality Products) and
1.9 g of the emulsifier polyoxyethylene(4)lauryl alcohol (Brij 30, from ICI) are placed into a vessel and dissolved in ethanol by stirring. Subsequently,
30.8 g of the film former ethyl cellulose are slowly added thereto under stirring. Thereafter,
14.5 g of the permeation enhancer oleic acid diethanolamide and
14.5 g of the permeation enhancer lauric acid hexyl ester are added and homogenised;
2.5 g of estradiol and
5.0 g of norethindrone acetate
   are added to the homogenised mass. Subsequently, this is stirred until the active substances are dissolved. By adding ethanol, the active substance content is adjusted to 50.0%.

The active substance-containing adhesive solution thus obtained is coated onto the backing layer (Hostaphan RN 23, from Mitsubishi), so that after drying an active agent-containing reservoir results which has a weight per unit area of 80-90 g/m². This layer is covered with a detachable protective layer (Hostaphan RN 100, vapour-deposited with aluminium on one side and siliconised on both sides). Individual patches are punched out of the laminate thus obtained.

### EXAMPLES 2 AND 3

Examples 2 and 3 too were prepared in the same manner as described above; their composition, like that of Example 1, can be seen from Table 1.

TABLE 1

| | Composition (%-wt) | | | | | |
|---|---|---|---|---|---|---|
| | Plastone | Ethyl cell. | Brij | | | Permeation |
| Example | S-630 | N50NF | 30 | Oes | NeA | enhancer |
| 1 | 30.8 | 30.8 | 1.9 | 2.5 | 5.0 | 14.5% OD, |
| | | | | | | 14.5% Cetiol A |
| 2 | 30.8 | 30.8 | 1.9 | 2.5 | 5.0 | 14.5% COD, |
| | | | | | | 14.5% Cetiol A |
| 3 | 30.8 | 30.8 | 1.9 | 2.5 | 5.0 | 14.5% COD, |
| | | | | | | 14.5% Eutanol G |

Explanation of the abbreviations:
Oes = estradiol
NeA = norethindrone acetate
OD = Comperlan OD = lauric acid diethanolamide
COD = Comperlan COD = coconut fatty acid diethanolamide
Cetiol A = lauric acid hexyl ester
Eutanol G = 2-octyl dodecanol
Ethyl cell. = ethyl cellulose

Examples 4, 5 and 6
were prepared in a corresponding manner and have the
following composition:
Composition (%-wt)

| | | Ethyl | | | | |
|---|---|---|---|---|---|---|
| Exam- | Plasdone | cellulose | Brij | Durotak | Oes/ | Permeation |
| ple | S-630 | N50 NF | 30 | 387-2287 | NeA | enhancer |
| 4 | 30.8 | 25.8 | 1.9 | 5.0 | * | 14.5% |
| | | | | | | Comperlan |
| | | | | | | OD |
| | | | | | | 14.5% |
| | | | | | | Cetiol A |
| 5 | 20.8 | 20.8 | 1.9 | 20.0 | * | 14.5% |
| | | | | | | Comperlan |
| | | | | | | OD |
| | | | | | | 14.5% |
| | | | | | | Cetiol A |
| 6 | 20.8 | 20.8 | 1.9 | 29.0 | * | 10.0% |
| | | | | | | Comperlan |
| | | | | | | OD |
| | | | | | | 10.0% |
| | | | | | | Cetiol A |

*The hormone content in Examples 4 to 6 corresponds to that of Examples
1 to 3 (Oes/NeA combination;
2.5%-wt Oes und 5.0%-wt NeA; s. Tab. 1).

### Durotak 387-2287: acrylate/methacrylate-vinylacetate Copolymer

To measure the human skin permeation, the skin is clamped into a Franz cell. An estrogen- and/or gestagen-containing patch of a surface area of 1.539 cm² is stuck onto the skin, and the active substance delivery is measured at 37° C. (acceptor medium: 0.9% sodium chloride solution with 0.1% NaN₃).

The results are listed in Table 2 (Examples 1-3), respectively in Table 3 (Examples 4-6).

Compared to Evorel Conti, the estradiol flux [μg/cm²·h] in Example 2 and 3 can be increased 3.6-fold, respectively 3.9-fold, and the norethindrone acetate flux 3.2-fold, respectively 3.9-fold. This means that the surface area of the TTS, which in the case of Evorel Conti is 16 cm², can be reduced to 4 cm² in the TTSs according to the present invention.

In addition, the transdermal therapeutic systems according to the invention are completely free from recrystallisation phenomena whereas with Evorel Conti there is a tendency for the active substance to crystallise.

The water absorptivity of Examples 1 and 2 and of the commercial product Evorel Conti was measured as follows. The TTSs were weighed and hung for one week in a saturated water vapour atmosphere inside a thin layer chromatography chamber. After removing the TTSs, their water content was determined by coulometric titration according to Karl Fischer. The results are shown in Table 4.

As the results of Table 4 show, the water content of the TTSs prepared according to Examples 1 and 2 is 28.2% and 36.7%, respectively, whereas Evorel Conti absorbs only 10% of water and, under these conditions, has a tendency towards increased active substance crystallisation. The patterns according to Example 1 and 2 by contrast show no crystallisation phenomena even under moist conditions.

TABLE 4

| | Water Absorptivity (%) |
|---|---|
| Example 1 | 28.3 |
| Example 2 | 36.7 |
| Evorel Conti | 10.0 |

The increased water absorption of the TTSs according to the present invention obviously leads to an increase in the solubility of the active substance(s) in the active substance-containing reservoir, and thereby surprisingly to an increase in the active substance release.

TABLE 2

| | Human Skin Permeation (μg/16 cm²) | | | | | | | | | | | | Content | |
|---|---|---|---|---|---|---|---|---|---|---|---|---|---|---|
| | Estradiol | | | | | | Norethindrone Acetate | | | | | | mg/16 cm² | |
| | 8 h | 24 h | 32 h | 48 h | 72 h | Flux | 8 h | 24 h | 32 h | 48 h | 72 h | Flux | Oes | NeA |
| Example 1 | 32 | 157 | 237 | 395 | 635 | 0.589 | 49 | 187 | 296 | 474 | 770 | 0.704 | 2.86 | 5.97 |
| Example 2 | 25 | 151 | 237 | 410 | 664 | 0.624 | 46 | 216 | 339 | 565 | 886 | 0.820 | 2.91 | 6.08 |
| Example 3 | 16 | 74 | 110 | 179 | 296 | 0.273 | 28 | 106 | 162 | 269 | 437 | 0.399 | 3.02 | 6.08 |
| Evorel | 23 | 78 | 103 | 144 | 214 | 0.187 | 28 | 90 | 125 | 179 | 272 | 0.238 | | |
| ContiP | | | | | | | | | | | | | | |

Flux: [μg/cm² · h]

TABLE 3
Human Skin Penetration (μg/16 cm²)

| | Estradiol | | | | | | Norethindrone Acetate | | | | | |
|---|---|---|---|---|---|---|---|---|---|---|---|---|
| | 8 h | 24 h | 32 h | 48 h | 72 h | 24-48 h | 8 h | 24 h | 32 h | 48 h | 72 h | 24-48 h |
| Example 4 | 13 | 75 | 112 | 195 | 342 | 120 | 27 | 114 | 178 | 296 | 467 | 182 |
| Example 5 | 15 | 90 | 134 | 227 | 392 | 137 | 28 | 119 | 186 | 310 | 491 | 191 |
| Example 6 | 30 | 132 | 189 | 306 | 483 | 174 | 39 | 147 | 219 | 350 | 518 | 203 |
| Evorel* | 12 | 40 | 50 | 73 | 125 | 33 | 20 | 62 | 88 | 130 | 189 | 68 |
| 8 J 124 Z | | | | | | | | | | | | |
| MRO-K* | 19 | 64 | 85 | 126 | 208 | 62 | 35 | 114 | 162 | 242 | 357 | 128 |
| (7/03948/ | | | | | | | | | | | | |
| 0) | | | | | | | | | | | | |

*Comparison examples

1. Transdermal therapeutic system in the form of a plaster, for the controlled delivery of estrogens and/or gestagens to human or animal skin, said system having a backing layer, an active substance-containing, mono- or multilayer reservoir connected thereto, and a detachable protective layer, characterized in that the active substance-containing reservoir contains vinyl pyrrolidone-vinyl acetate copolymer and cohesion-enhancing substances, said cohesion-enhancing substances being selected from the group consisting of polyethylene, polypropylene, salts of ethylene-methacrylic acid copolymers, polystyrene, polybutene, polyisobutylene, styrene-butadiene-styrene block polymers, polyvinyl chloride, polyvinylidene chloride, polyurethane, polyamide, polysulfone, polyvinyl acetate, polyvinyl alcohol, polyethylene glycol, polyoxyethylene, polyvinyl butyral, and ethylene-vinyl acetate copolymers.

2. Transdermal therapeutic system according to claim 1, characterized in that the content of vinyl pyrrolidone-vinyl acetate copolymer in the active substance-containing reservoir is 1.0 to 95.0%-wt, preferably 2.0 to 90.0%-wt.

3. Transdermal therapeutic system according to claim 1 or 2, characterized in that the vinyl pyrrolidone/vinyl acetate ratio of the vinyl pyrrolidone-vinyl acetate copolymer is 15:85 to 85:15, preferably 20:80 to 70:30.

4. Transdermal therapeutic system according to claim 1, characterized in that the content of cohesion-improving substances in the active substance-containing reservoir is 1.0 to 60.0%-wt, preferably 2.0 to 50.0%-wt.

5. Transdermal therapeutic system according to claim 1, characterized in that the active substance-containing reservoir contains 3.0 to 70.0%-wt, preferably 5.0 to 60.0%-wt, of ethylene-vinyl acetate copolymer.

6. Transdermal therapeutic system according to claim 5, characterized in that the vinyl acetate content of the ethylene-vinyl acetate copolymer is 15 to 50%-wt, preferably 18 to 40%-wt.

7. Transdermal therapeutic system according claim 1, characterized in that the active substance-containing reservoir consists of at least two spatially separated parts.

8. Transdermal therapeutic system according to claim 7, characterized in that one spatially separated part of the active substance-containing reservoir contains an estrogen, and that the other spatially separated part contains a gestagen, either alone or in combination with an estrogen.

9. Transdermal therapeutic system according to claim 1, characterized in that the estrogen concentration in the active substance-containing reservoir is 0.5 to 10.0%-wt, preferably 1 to 5.0%-wt, and that the gestagen concentration is 0.5 to 20.0%-wt, preferably 1.0 to 10.0%-wt.

10. Transdermal therapeutic system according to claim 1, characterized in that the active substance-containing reservoir contains tack-enhancing resins in a concentration of 5.0 to 70.0%-wt, preferably 10.0 to 60.0%-wt.

11. Transdermal therapeutic system according to claim 1, characterized in that the active substance-containing reservoir contains skin permeation-enhancing substances in a concentration of 1.0 to 50.0%-wt, preferably 3.0 to 45.0%-wt.

12. Transdermal therapeutic system according to claim 1, characterized in that the active substance-containing reservoir contains emulsifiers and/or plasticizers and/or antioxidants in a respective concentration of up to 25.0%-wt, preferably 1.0 to 15.0%-wt.

13. Transdermal therapeutic system according to claim 1, characterized in that the active substance-containing reservoir contains fillers.

14. Transdermal therapeutic system according to claim 1, characterized in that the active substance-containing reservoir is made up of two or more layers.

15. Transdermal therapeutic system according to claim 14, characterized in that the individual layers of the active substance-containing reservoir contain different active substances and/or differ from each other in terms of their active substance concentration and/or their composition.

16. Transdermal therapeutic system according to claim 14 or 15, characterized in that between the layers of the active substance-containing reservoir there is interposed a flat body, preferably a membrane, a film, a textile fabric, a nonwoven fabric, or a woven fabric.

17. Transdermal therapeutic system according to claim 1, characterized in that the active substance-containing reservoir has a layer thickness of 0.02 mm to 0.5 mm, preferably 0.03 to 0.3 mm.

18. Transdermal therapeutic system according to claim 1, characterized in that the active substance-containing reservoir is provided with a pressure sensitive adhesive layer and/or a pressure-sensitive adhesive margin.

19. Process for the production of a transdermal therapeutic system according to claim 1, comprising the following steps:
preparing a solution or suspension containing the components of the active substance-containing reservoir, and
coating this solution or suspension on a flat support.

20. Process for the production of a transdermal therapeutic system according to claim 1, comprising the following steps:
preparing a melt containing the components of the active substance-containing reservoir, and
coating this melt on a flat support.

21. Use of the transdermal therapeutic system according to claim 1 for therapeutic purposes in human medicine, preferably for hormone substitution, for the prophylaxis and treatment of climacteric complaints, as well as for the prophylaxis of osteoporosis.

22. Use of the transdermal therapeutic system according to claim 1 for hormonal contraception.
